(19) United States
(12) Patent Application Publication
(10) Pub. No.: US 2014/0172057 A1
(43) Pub. Date: Jun. 19, 2014

(54) SYSTEMS AND METHODS FOR MAKING AND USING PADDLE LEADS OF ELECTRICAL STIMULATION SYSTEMS

(71) Applicant: BOSTON SCIENTIFIC NEUROMODULATION CORPORATION, VALENCIA, CA (US)

(72) Inventor: William George Orinski, Reno, NV (US)

(73) Assignee: BOSTON SCIENTIFIC NEUROMODULATION CORPORATION, VALENCIA, CA (US)

(21) Appl. No.: 14/101,882

(22) Filed: Dec. 10, 2013

Related U.S. Application Data
(60) Provisional application No. 61/737,534, filed on Dec. 14, 2012.

(51) Int. Cl. A61N 1/05; H05K 3/36

(52) U.S. Cl. CPC A61N 1/05; H05K 3/36
USPC 607/116; 29/830

(57) ABSTRACT

An electrical stimulation lead includes a paddle body with micro-circuit assemblies having micro-circuits laminated between electrically-nonconductive substrates. The micro-circuits have first end portions and opposing second end portions. Electrodes are electrically coupled to the first end portions of the micro-circuits. Distal end portions of one or more lead bodies are coupled to the paddle body. Terminals are disposed along proximal end portions of the one or more lead bodies. Lead-body conductors are coupled to the terminals and extend along the one or more lead bodies to distal end portions of the one or more lead bodies. The lead-body conductors are attached to the second end portions of the micro-circuits to electrically couple the terminals to the electrodes.

### CROSS-REFERENCE TO RELATED APPLICATIONS

This application claims the benefit under 35 U.S.C. §119(e) of U.S. Provisional Patent Application Ser. No. 61/737,534 filed Dec. 14, 2012, which is incorporated herein by reference.

### FIELD

The present invention is directed to the area of implantable electrical stimulation systems and methods of making and using the systems. The present invention is also directed to implantable electrical stimulation leads having paddle bodies that include micro-circuits formed along micro-circuit assemblies, as well as methods of making and using the leads, paddle bodies, micro-circuits, micro-circuit assemblies, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, an electrical stimulation lead includes a paddle body having a proximal end portion, a distal end portion, a first major surface, and an opposing second major surface. The paddle body includes a plurality of micro-circuit assemblies. Each of the plurality of micro-circuit assemblies includes a first electrically-nonconductive substrate, a second electrically-nonconductive substrate, and a plurality of micro-circuits laminated between the first electrically-nonconductive substrate and the second electrically-nonconductive substrate. The plurality of micro-circuits each has a first end portion and an opposing second end portion. The second end portions of the plurality of micro-circuits are disposed at the proximal end portion of the paddle body. A plurality of electrodes is disposed on the paddle body. The plurality of electrodes is electrically coupled to the first end portions of the plurality of micro-circuits. At least one lead body has a distal end portion, a proximal end portion, and a longitudinal length. The distal end portions of each of the at least one lead body is coupled to the proximal end portion of the paddle body. A plurality of terminals is disposed along the proximal end portion of each of the at least one lead body. A plurality of lead-body conductors is coupled to the plurality of terminals and extends along the at least one lead body to the distal end portion of the at least one lead body. The plurality of lead-body conductors is attached to the second end portions of the plurality of micro-circuits and electrically couples the plurality of terminals to the plurality of electrodes.

In another embodiment, a method of forming a paddle lead includes forming a plurality of micro-circuit assemblies. Each of the plurality of micro-circuit assemblies includes a plurality of micro-circuits laminated between electrically-nonconductive substrates. Each of the plurality of micro-circuits has a first end portion and an opposing second end portion. A plurality of electrodes is mechanically coupled to the plurality of micro-circuit assemblies. The plurality of electrodes is electrically coupled to the first end portions of the plurality of micro-circuits. The plurality of micro-circuit assemblies and a carrier are coupled together to form a paddle body. A plurality of lead-body conductors is extended along at least one lead body. The second end portions of the plurality of micro-circuits are attached to the lead-body conductors. The paddle body is mechanically coupled to the at least one lead body.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a lead electrically coupled to a control module, according to the invention;

FIG. 2A is a schematic view of one embodiment of the control module of FIG. 1 configured and arranged to electrically couple to an elongated device, according to the invention;

FIG. 2B is a schematic view of one embodiment of a lead extension configured and arranged to electrically couple the elongated device of FIG. 2A to the control module of FIG. 1, according to the invention;

FIG. 3A is a schematic top perspective view of one embodiment of distal portion of a paddle lead, the distal portion of the paddle lead including a paddle body disposed along distal end portions of multiple lead bodies, the paddle body including electrodes coupled to micro-circuits, according to the invention;

FIG. 3B is a schematic transverse cross-sectional view of one embodiment of the multiple lead bodies shown in FIG. 3A, according to the invention;

FIG. 4A is a schematic top perspective view of one embodiment of a first coupon that includes at least some of the micro-circuits of the paddle body of FIG. 3A, according to the invention;

FIG. 4B is a schematic top perspective view of one embodiment of a second coupon that includes at least some of the micro-circuits of the paddle body of FIG. 3A, according to the invention;

FIG. 5A is a schematic top perspective view of one embodiment of the coupon of FIG. 4A and a first top insulative substrate configured and arranged for disposing over a first major surface of the coupon, according to the invention;

FIG. 5B is a schematic top perspective view of one embodiment of the coupon of FIG. 4B and a second top insulative substrate configured and arranged for disposing over a first major surface of the coupon, according to the invention;

FIG. 6A is a schematic perspective top view of one embodiment of the coupon of FIG. 4A and a first bottom insulative substrate configured and arranged for disposing over a second major surface of the coupon, according to the invention;

FIG. 6B is a schematic top perspective view of one embodiment of the coupon of FIG. 4B and a second bottom insulative substrate configured and arranged for disposing over a second major surface of the coupon, according to the invention;

FIG. 7A is a schematic top perspective view of one embodiment of a first micro-circuit assembly formed by laminating the first coupon of FIG. 4A between the first top insulative substrate of FIG. 5A and the first bottom insulative substrate of FIG. 6A, according to the invention;

FIG. 7B is a schematic top perspective view of one embodiment of a second micro-circuit assembly formed by laminating the second coupon of FIG. 4B between the second top insulative substrate of FIG. 5B and the second bottom insulative substrate of FIG. 6B, according to the invention;

FIG. 8A is a schematic top perspective view of one embodiment of the first micro-circuit assembly of FIG. 7A, where conductor tie bars formerly coupling together adjacent micro-circuits of the first micro-circuit assembly have been removed to electrically isolate adjacent micro-circuits from one another, according to the invention;

FIG. 8B is a schematic top perspective view of one embodiment of the second micro-circuit assembly of FIG. 7B, where conductor tie bars formerly coupling together adjacent micro-circuits of the second micro-circuit assembly have been removed to electrically isolate adjacent micro-circuits from one another, according to the invention;

FIG. 9A is a schematic top perspective view of one embodiment of the first micro-circuit assembly of FIG. 8A and multiple first electrodes, where the first electrodes are configured and arranged for disposing over risers formed at first ends of micro-circuits of the first micro-circuit assembly, according to the invention;

FIG. 9B is a schematic top perspective view of one embodiment of the second micro-circuit assembly of FIG. 8B and multiple second electrodes, where the second electrodes are configured and arranged for disposing over risers formed at first ends of micro-circuits of the second micro-circuit assembly, according to the invention;

FIG. 10A is a schematic top perspective view of one embodiment of the first micro-circuit assembly and the first electrodes of FIG. 9A, where the first electrodes are mechanically coupled to the first micro-circuit assembly, where first ends of micro-circuits of the first micro-circuit assembly are electrically coupled to the first electrodes, and where second ends of the micro-circuits are configured to attach to conductors of the one or more lead bodies of FIG. 3A, according to the invention;

FIG. 10B is a schematic top perspective view of one embodiment of the second micro-circuit assembly and the second electrodes of FIG. 9B, where the second electrodes are mechanically coupled to the second micro-circuit assembly, where first ends of micro-circuits of the second micro-circuit assembly are electrically coupled to the second electrodes, and where second ends of the micro-circuits are configured to attach to conductors of the one or more lead bodies of FIG. 3A, according to the invention;

FIG. 11A is a schematic bottom perspective view of one embodiment of the first micro-circuit assembly and the first electrodes of FIG. 9A, where the first electrodes are disposed over risers along a first major surface of the first micro-circuit assembly, and where the first electrodes are electrically coupled to micro-circuits of the first micro-circuit assembly along a second major surface of the first micro-circuit assembly, according to the invention;

FIG. 11B is a schematic bottom perspective view of one embodiment of the second micro-circuit assembly and the second electrodes of FIG. 9B, where the second electrodes are disposed over risers along a first major surface of the second micro-circuit assembly, and where the second electrodes are electrically coupled to micro-circuits of the second micro-circuit assembly along a second major surface of the second micro-circuit assembly, according to the invention;

FIG. 12A is a schematic top perspective view of one embodiment of the first and second micro-circuit assemblies of FIG. 10A, where the second micro-circuit assembly is configured and arranged for disposing over a portion of a first major surface of the first micro-circuit assembly, according to the invention;

FIG. 12B is a schematic top perspective view of one embodiment of the second micro-circuit assembly of FIG. 10A coupled to a first major surface of the first micro-circuit assembly of FIG. 10A to form a composite micro-circuit assembly, according to the invention;

FIG. 13 is a schematic top perspective view of one embodiment of the composite micro-circuit assembly of FIG. 12B and a molded carrier configured and arranged for disposing along a second major surface of the composite micro-circuit assembly, according to the invention;

FIG. 14 is a schematic top perspective view of one embodiment of a pre-paddle body formed by coupling the pre-molded carrier of FIG. 13 to the composite micro-circuit assembly of FIG. 13, according to the invention;

FIG. 15 is a schematic side view of one embodiment of the paddle body of FIG. 3A formed by over-molding the pre-paddle body of FIG. 14, the paddle body including removable tooling bosses for facilitating the over-molding process, according to the invention; and

FIG. 16 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to implantable electrical stimulation leads having paddle bodies that include micro-circuits formed along micro-circuit assemblies, as well as methods of making and using the leads, paddle bodies, micro-circuits, micro-circuit assemblies, and electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364,278; and U.S. Patent Application Publication No. 2007/0150036, all of which are incorporated herein by reference.

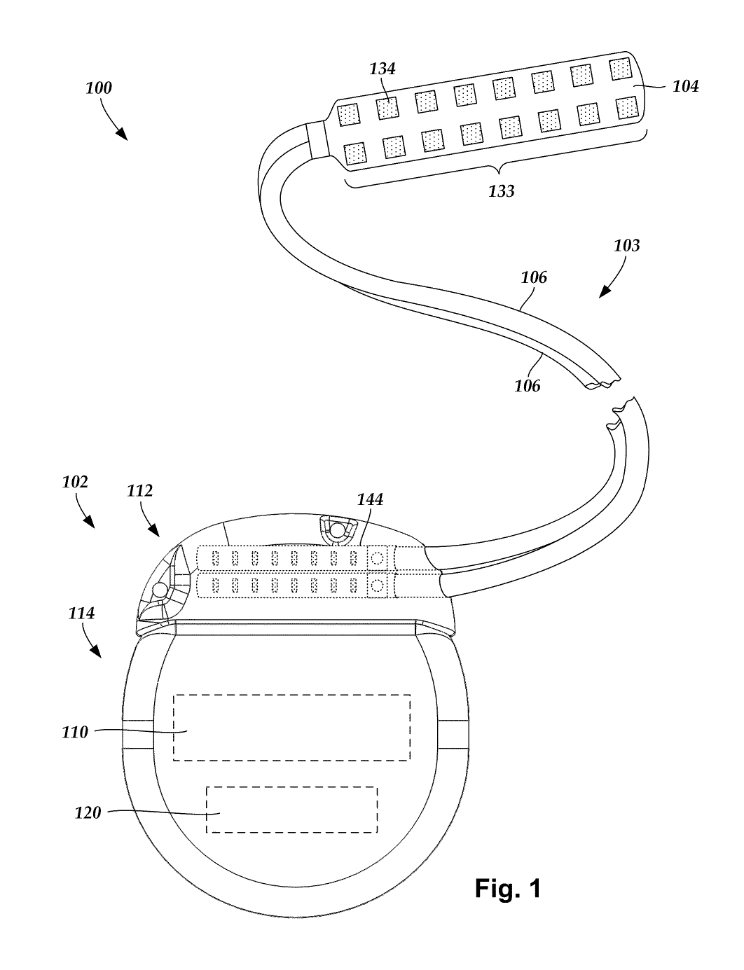

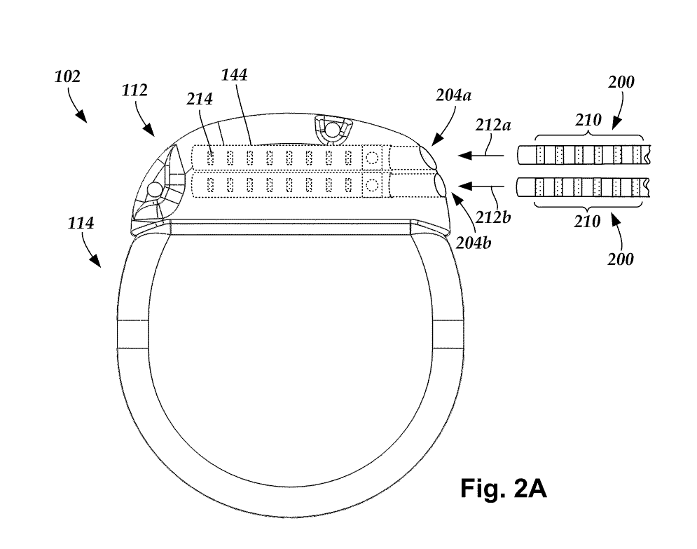

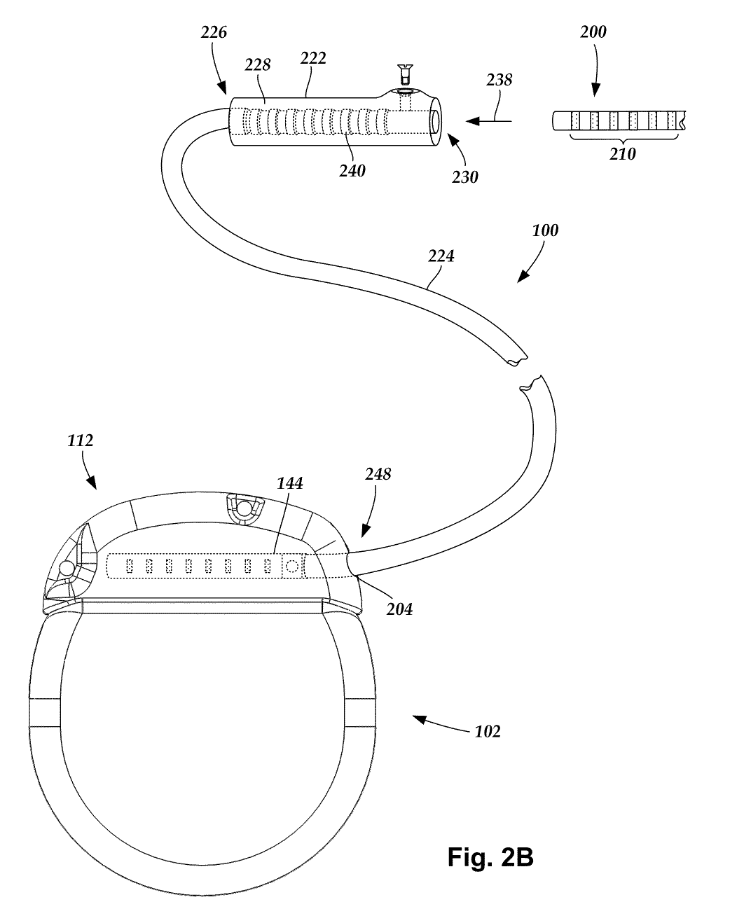

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a paddle body 104 and one or more lead bodies 106. In FIG. 1, the lead 103 is shown having two lead bodies 106. It will be understood that the lead 103 can include any suitable number of lead bodies including, for example, one, two, three, four, five, six, seven, eight or more lead bodies 106. An array of electrodes 133, such as electrode 134, is disposed on the paddle body 104, and an array of terminals (e.g., 210 in FIG. 2A-2B) is disposed along each of the one or more lead bodies 106.

The lead 103 can be coupled to the control module 102 in any suitable manner. In FIG. 1, the lead 103 is shown coupling directly to the control module 102. In at least some other embodiments, the lead 103 couples to the control module 102 via one or more intermediate devices. For example, in at least some embodiments one or more lead extensions 224 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

The control module 102 typically includes a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

The electrical stimulation system or components of the electrical stimulation system, including the paddle body 104, the one or more of the lead bodies 106, and the control module 102, are typically implanted into the body of a patient. The electrical stimulation system can be used for a variety of applications including, but not limited to neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

Any suitable number of electrodes 134 can be disposed on the paddle body including, for example, four, five, six, seven, eight, nine, ten, eleven, twelve, fourteen, sixteen, twenty-four, thirty-two, or more electrodes 134. The electrodes 134 can be disposed on the paddle body 104 in any suitable arrangement. In FIG. 1, the electrodes 134 are arranged into two columns, where each column has eight electrodes 134.

The electrodes of the paddle body 104 are typically disposed in, or separated by, a non-conductive, biocompatible material such as, for example, silicone, polyurethane, polyetheretherketone (“PEEK”), epoxy, and the like or combinations thereof. The paddle body 104 and the one or more lead bodies 106 may be formed in the desired shape by any process including, for example, molding (including injection molding), casting, and the like. The non-conductive material typically extends from the paddle body 104 to the proximal end of each of the one or more lead bodies 106. The non-conductive, biocompatible material of the paddle body 104 and the one or more lead bodies 106 may be the same or different. The paddle body 104 and the one or more lead bodies 106 may be a unitary structure or can be formed as two separate structures that are permanently or detachably coupled together.

Terminals (e.g., 210 in FIGS. 2A-2B) are typically disposed along the proximal end of the one or more lead bodies 106 of the electrical stimulation system 100 (as well as any splitters, lead extensions, adaptors, or the like) for electrical connection to corresponding connector contacts (e.g., 214 in FIGS. 2A-2B). The connector contacts are disposed in connectors (e.g., 144 in FIGS. 1-2B; and 222 FIG. 2B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors may extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may also be one or more lumens (not shown) that open at, or near, the distal end of the one or more lead bodies 106, for example, for infusion of drugs or medication into the site of implantation of the one or more lead bodies 106. In at least one embodiment, the one or more lumens may be flushed continually, or on a regular basis, with saline, epidural fluid, or the like. In at least some embodiments, the one or more lumens can be permanently or removably sealable at the distal end.

FIG. 2A is a schematic side view of one embodiment of a proximal end of one or more elongated devices 200 configured and arranged for coupling to one embodiment of the control module connector 144. The one or more elongated devices may include, for example, one or more of the lead bodies 106 of FIG. 1, one or more intermediate devices (e.g., a splitter, the lead extension 224 of FIG. 2B, an adaptor, or the like or combinations thereof), or a combination thereof.

The control module connector 144 defines at least one port into which a proximal end of the elongated device 200 can be inserted, as shown by directional arrows 212a and 212b. In FIG. 2A (and in other figures), the connector housing 112 is shown having two ports 204a and 204b. The connector housing 112 can define any suitable number of ports including, for example, one, two, three, four, five, six, seven, eight, or more ports.

The control module connector 144 also includes a plurality of connector contacts, such as connector contact 214, disposed within each port 204a and 204b.

When the elongated device 200 is inserted into the ports 204a and 204b, the connector contacts 214 can be aligned with a plurality of terminals 210 disposed along the proximal end(s) of the elongated device(s) 200 to electrically couple the control module 102 to the electrodes (134 of FIG. 1) disposed on the paddle body 104 of the lead 103. Examples of connectors in control modules are found in, for example, U.S. Pat. Nos. 7,244,150 and 8,224,450, which are incorporated by reference.

FIG. 2B is a schematic side view of another embodiment of the electrical stimulation system 100. The electrical stimulation system 100 includes a lead extension 224 that is configured and arranged to couple one or more elongated devices 200 (e.g., one of the lead bodies 106 of FIG. 1, a splitter, an adaptor, another lead extension, or the like or combinations thereof) to the control module 102. In FIG. 2B, the lead extension 224 is shown coupled to a single port 204 defined in the control module connector 144. Additionally, the lead extension 224 is shown configured and arranged to couple to a single elongated device 200. In alternate embodiments, the lead extension 224 is configured and arranged to couple to multiple ports 204 defined in the control module connector 144 (e.g., the ports 204a and 204b of FIG. 1), or to receive multiple elongated devices 200 (e.g., both of the lead bodies 106 of FIG. 1), or both.

A lead extension connector 222 is disposed on the lead extension 224. In FIG. 2B, the lead extension connector 222 is shown disposed at a distal end 226 of the lead extension 224. The lead extension connector 222 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (134 of FIG. 1) disposed along the lead (103 in FIG. 1).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the control module connector 144.

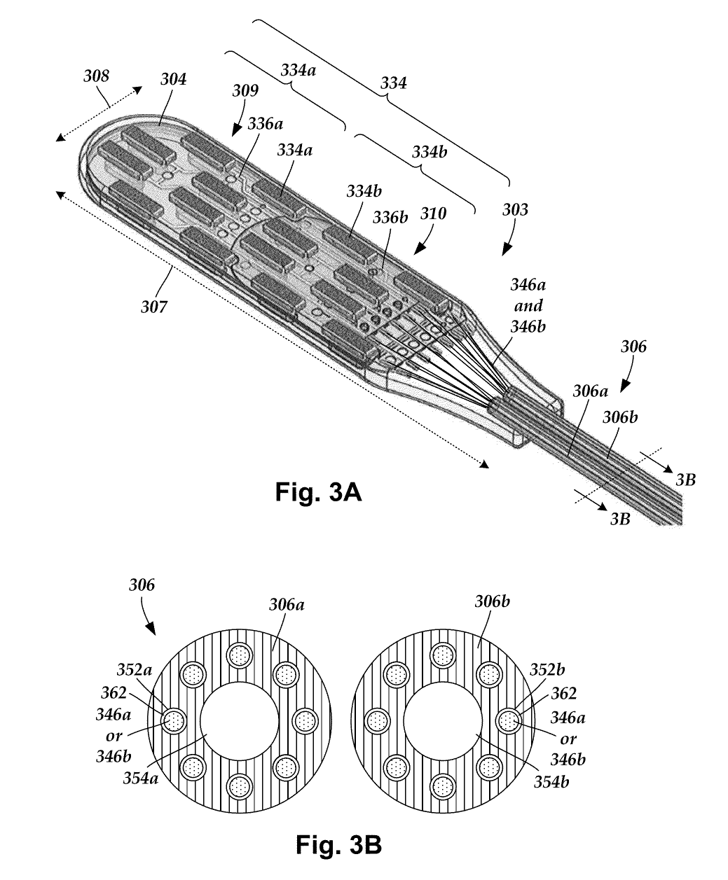

Turning to FIG. 3A, in at least some embodiments paddle bodies include one or more micro-circuits (e.g., conductive traces, or the like) in lieu of conventional electrically-conductive wires. The micro-circuits are disposed on micro-circuit assemblies (e.g., conductive traces laminated between electrically-nonconductive substrates). The micro-circuits may be attached to other conductors, such as conductive wires, extending along the lead body. It may be advantageous to utilize micro-circuits in paddle bodies to provide reliability and consistency in the manufacturing process of paddle bodies. Additionally, micro-circuits may simplify the manufacturing process and reduce costs associated therewith.

FIG. 3A is a schematic top perspective view of one embodiment of a distal portion of a paddle lead 303. The distal portion of the paddle lead 303 includes a paddle body 304 disposed along a distal end portion of one or more lead bodies 306. The paddle body 304 has a length 307, a width 308, a distal end portion 309, and a proximal end portion 310.

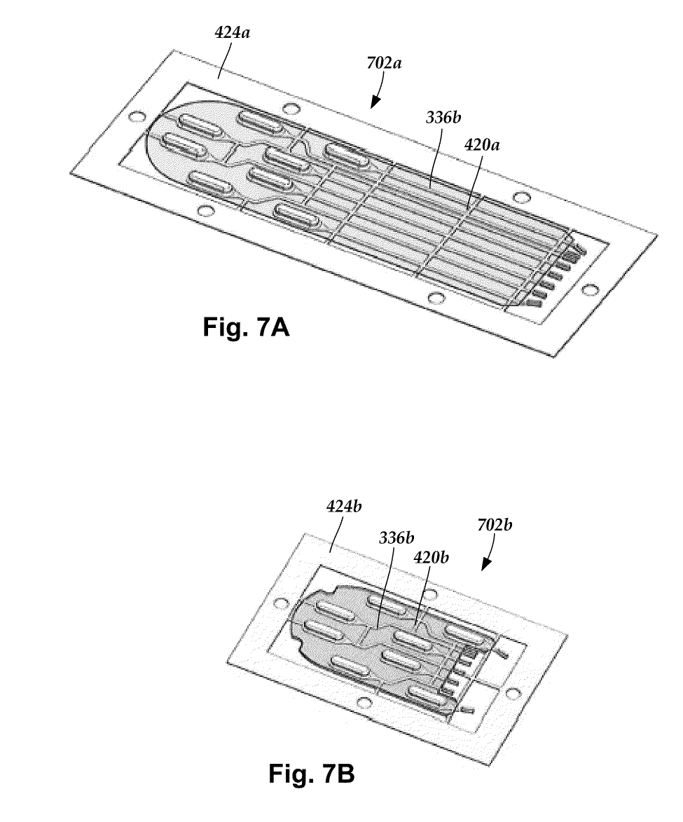

An array of electrodes 334 is disposed along the paddle body 304. In FIG. 3A, the electrodes 334 are shown divided into two groupings: distal electrodes, such as distal electrode 334a; and proximal electrodes, such as proximal electrode 334b. As will be discussed in more detail below, the distal electrodes 334a are associated with a first micro-circuit assembly (see e.g., 702a of FIG. 7A), and the proximal electrodes 334b are associated with a second micro-circuit assembly (see e.g., 702b of FIG. 7B).

Each of the electrodes 334 is electrically coupled to one or more micro-circuits, such as micro-circuits 336a and 336b. In at least some embodiments the micro-circuits 336a and 336b are each associated with a different micro-circuit assembly (see e.g., 702a of FIGS. 7A and 702b of FIG. 7B). In at least some embodiments, each of the electrodes 334 is individually coupled to a different micro-circuit 336a or 336b. In at least some embodiments, the distal electrodes 334a are coupled to the micro-circuits 336a, and the proximal electrodes 334b are coupled to the micro-circuits 336b.

The paddle body 304 is coupled at one end to the one or more lead bodies 306. In FIG. 3A, the paddle lead 303 is shown having two lead bodies 306a and 306b. It will be understood that the paddle lead 303 may include any suitable number of lead bodies 306 including, for example one, two, three, four, five, six, seven, eight, or more lead bodies 306. Conductors, such as conductive wires 346a and 346b, extend along the lead bodies 306 and attach to the micro-circuits 336a and 336b. It will be understood that the micro-circuits 336a may be attached exclusively to the conductive wires 346a, or exclusively to the conductive wires 346b, or to a combination of both the conductive wires 346a and 346b. Similarly, the micro-circuits 336b may be attached exclusively to the conductive wires 346a, or exclusively to the conductive wires 346b, or to a combination of both the conductive wires 346a and 346b.

In FIG. 3A, sixteen electrodes 334 are shown disposed along the paddle body 304. It will be understood that any suitable number of electrodes 334 can be disposed along the paddle body 304 including, for example, one, two, three, four, five, six, seven, eight, nine, ten, twelve, sixteen, twenty, twenty-four, thirty, thirty-two, thirty-six, forty-eight, or more. In FIG. 3A, the electrodes 334 are arranged along the paddle body 304 in four columns, where each column includes four electrodes 334. It will be understood that the electrodes 334 can be disposed along the paddle body 304 in any suitable arrangement. Optionally, the electrodes 334 of at least one column can be longitudinally offset from the electrodes 334 of at least one other column along the length 308 of the paddle body.

When the electrodes 334 are divided into multiple groupings, such as the distal electrodes 334a and the proximal electrodes 334b, the number of electrodes within each grouping can be equal to one another, or can be unequal to one another. In FIG. 3A, and in other figures, the number of distal electrodes 334a is equal to the number of proximal electrodes 334b.

FIG. 3B is a schematic transverse cross-sectional view of one embodiment of the lead bodies 306a and 306b. Each of the lead bodies 306a and 306b defines one or more conductor lumens, such as conductor lumens 352a and 352b, respectively, and stylet lumens 354a and 354b, respectively. The conductive wires 346a and 346b extend along the lead bodies 306a and 306b within the conductor lumens 352a and 352b. The conductive wires 346a and 346b may, optionally, be individually covered with insulation 362. This may be advantageous to prevent short-circuiting; for example, in embodiments when multiple conductive wires 346a or 346b extend along the same conductor lumen 352a or 352b.

It will be understood that the lead bodies 306a and 306b shown in FIG. 3B are one of many suitable arrangements that can be used for forming the lead bodies 306a and 306b. In alternate embodiments, the lead bodies 306a and 306b may, for example, not define a stylet lumen, or may define more (or fewer) conductor lumens from the embodiment of the lead bodies 306a and 306b shown in FIG. 3B. In at least some embodiments, the lead bodies 306a and 306b do not define any conductor lumens at all. In at least some embodiments, the lead body 306a has an arrangement that is different from the lead body 306b.

The paddle body 304 can be formed using one or more micro-circuit assemblies. The micro-circuit assemblies each include micro-circuits laminated between electrically-nonconductive substrates. In at least some embodiments, the micro-circuits of the micro-circuit assemblies are formed from framed coupons that include multiple individual electrically-conductive traces coupled to a frame and to one another. When multiple micro-circuit assemblies are used, a different subset of the electrodes is coupled to each micro-circuit assembly and the micro-circuit assemblies are coupled to one another in any suitable arrangement. For example, as shown in FIG. 3A the distal electrodes 334a are associated with a first micro-circuit assembly (see e.g., 702a of FIG. 7A), and the proximal electrodes 334b are associated with a second micro-circuit assembly (see e.g., 702b of FIG. 7B).

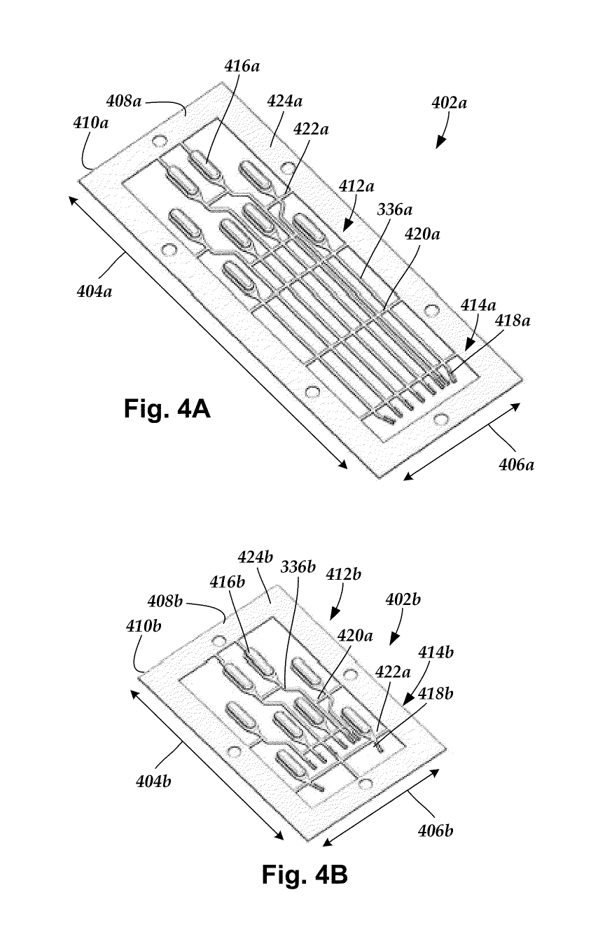

FIGS. 4A-15 describe one embodiment of forming the paddle body 304 using micro-circuit assemblies. In the embodiment shown in FIGS. 4A-15, two micro-circuit assemblies are used, where one of the micro-circuit assemblies is coupled to distal electrodes 334a, and the other of the micro-circuit assemblies is coupled to proximal electrodes 334b. The embodiment described in FIGS. 4A-15 additionally includes mechanically coupling the micro-circuit assemblies together and to a carrier; mechanically coupling the paddle body to lead bodies; and attaching the micro-circuits to conductors extending along the lead bodies.

FIG. 4A is a schematic top perspective view of one embodiment of a first coupon 402a suitable for use in the paddle body 304. FIG. 4B is a schematic top perspective view of one embodiment of a second coupon 402b suitable for use, along with the first coupon 402a, in the paddle body 304.

The first coupon 402a includes the micro-circuits 336a and the second coupon 402b includes the micro-circuits 336b. The micro-circuits 336a are coupled to one another by conductor tie bars 420a. Similarly, the micro-circuits 336b are coupled to one another by conductor tie bars 420b. In at least some embodiments, the conductor tie bars 420a and 420b are rigid to maintain a constant spacing between adjacent micro-circuits. Optionally, the first coupon 402a and the second coupon 402b include frames 424a and 424b, respectively. In which case, the micro-circuits 336a may be coupled to the frame 424a via one or more frame tie bars 422a. Similarly, the micro-circuits 336b may be coupled to the frame 424b via one or more frame tie bars 422b. In at least some embodiments, the frame tie bars 422a and 422b are rigid to maintain a constant spacing between adjacent micro-circuits and between the frame and the micro-circuits.

In at least some embodiments, the micro-circuits 336a are formed as a plurality of individual conductive traces that extend generally parallel to one another along the coupon 402a. Similarly, in at least some embodiments the micro-circuits 336b are formed as a plurality of individual conductive traces that extend generally parallel to one another along the coupon 402b. The micro-circuits 336a and 336b can be formed from any electrically-conductive material suitable for implantation including, for example, metals, alloys, conductive polymers, conductive carbon, or the like. In at least some embodiments, the micro-circuits 336a and 336b are formed from MP35N.

It may be advantageous for the micro-circuits 336a and 336b to be coupled to one another by one or more conductor tie bars 420a and 420b, respectively, to maintain a constant spacing between adjacent micro-circuits. In embodiments with frames 424a and 424b, it may be advantageous for the micro-circuits 336a and 336b to be coupled to the frame by one or more frame tie bars 422a and 422b, respectively, to further maintain a constant spacing between adjacent micro-circuits, and also to facilitate handling of the coupons 402a and 402b. It may further be advantageous to use conductor tie bars 420a and 420b and frame tie bars 422a and 422b to facilitate manufacture of the coupons 402a and 402b. For example, in at least some embodiments the coupons 402a and 402b are stamped.

The micro-circuits 336a each have a first end portion 412a and an opposing second end portion 414a. In at least some embodiments, risers, such as riser 416a, are disposed in proximity to the first end portions 412a of the micro-circuits 336a. In at least some embodiments, the risers 416a are configured and arranged to receive the electrodes 334a. The second end portions 414a of the micro-circuits 336a are configured and arranged to attach to the lead-body conductors 346a or 346b. In at least some embodiments, the second end portions 414a of the micro-circuits 336a each include an electrical connector, such as electrical connector 418a, for facilitating attachment of the micro-circuits 336a to the conductive wires 346a or 346b.

Similarly, the micro-circuits 336b each have a first end portion 412b and an opposing second end portion 414b. In at least some embodiments, risers, such as riser 416b, are disposed in proximity to the first end portions 412b of the micro-circuits 336b. In at least some embodiments, the risers 416b are configured and arranged to receive the electrodes 334b. The second end portions 414b of the micro-circuits 336b are configured and arranged to attach to the lead-body conductors 346a or 346b. In at least some embodiments, the second end portions 414b of the micro-circuits 336a each include an electrical connector, such as electrical connector 418b, for facilitating attachment of the micro-circuits 336b to the lead-body conductors 346a or 346b.

The coupons 402a and 402b have first major surfaces 408a and 408b, respectively, and opposing second major surfaces 410a and 410b, respectively. In at least some embodiments, the risers 416a and 416b extend outwardly from the first major surfaces 408a and 408b, respectively, of the coupons 402a and 402b, respectively. As will be discussed in more detail below, in at least some embodiments the risers 416a and 416b provide locations on the first and second coupons over which the electrodes 334 are disposed. Accordingly, in at least some embodiments the risers 416a and 416b are collectively arranged into a physical pattern that matches the arrangement of the electrodes 434 on the paddle body 304.

The micro-circuits 336a and 336b may be attached to the lead-body conductors 346a and 346b, respectively, in a number of different ways. In at least some embodiments, the electrical connectors 418a and 418b are configured and arranged for welding to the lead-body conductors 346a and 346b, respectively. In at least some embodiments, the electrical connectors 418a and 418b are configured and arranged for crimping to the lead-body conductors 346a and 346b, respectively. In at least some embodiments, the electrical connectors 418a and 418b are configured and arranged for welding and crimping to the lead-body conductors 346a and 346b, respectively. In at least some embodiments, the electrical connectors 418a and 418b include one or more cable crimp lugs.

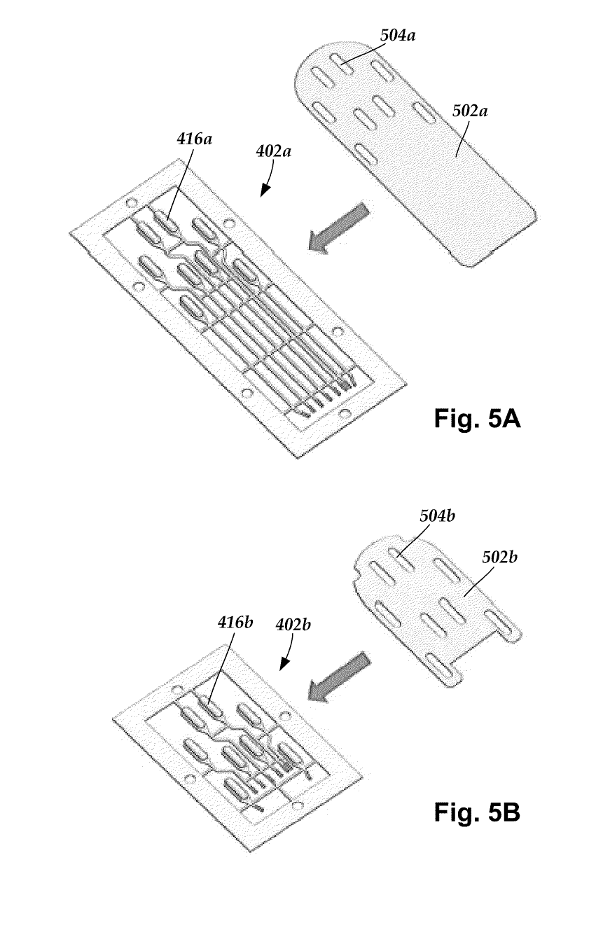

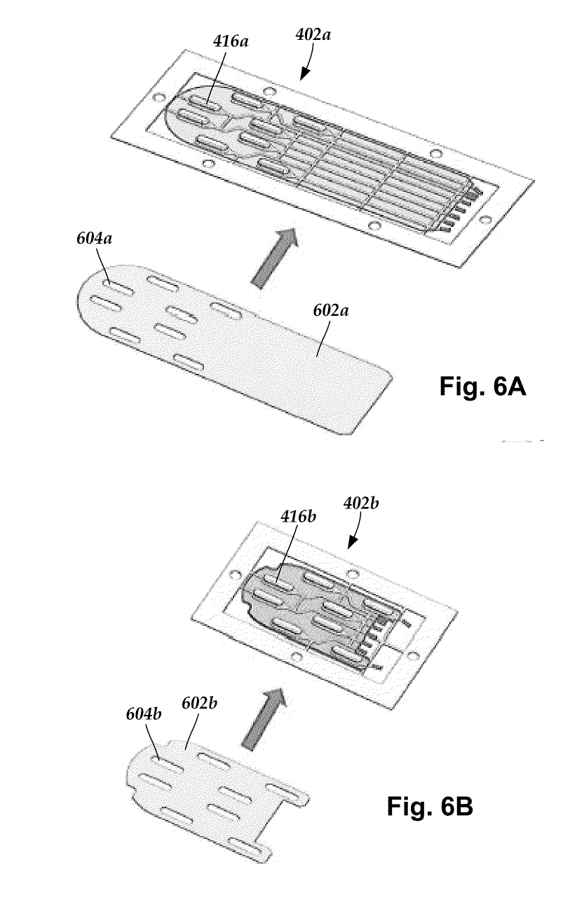

Turning to FIGS. 5A-6B, in at least some embodiments the coupons 402a and 402b are laminated between sheets of electrically-insulative material. FIG. 5A is a schematic top perspective view of one embodiment of the first coupon 402a and a first top insulative substrate 502a configured and arranged for disposing over the first major surface 408a of the first coupon 402a. FIG. 5B is a schematic top perspective view of one embodiment of the second coupon 402b and a second top insulative substrate 502b configured and arranged for disposing over the first major surface 408b of the second coupon 402a.

FIG. 6A is a schematic top perspective view of one embodiment of the first coupon 402a and a first bottom insulative substrate 602a configured and arranged for disposing over the second major surface 410a of the first coupon 402a. FIG. 6B is a schematic top perspective view of one embodiment of the second coupon 402b and a second bottom insulative substrate 602b configured and arranged for disposing over the second major surface 410b of the second coupon 402b.

In at least some embodiments, the first top insulative substrate 502a defines cutouts, such as cutout 504a, positioned to coincide with the positioning of corresponding risers 416a of the first coupon 402a such that, when the first top insulative substrate 502a is disposed over the first major surface 410a of the first coupon 402a, the risers 416a extend through the cutouts 504a. In at least some embodiments, the second top insulative substrate 502b similarly defines cutouts 504b positioned to coincide with the positioning of corresponding risers 416b of the second coupon 402b. In at least some embodiments, the first bottom insulative substrate 602a defines cutouts 604a positioned to coincide with the positioning of the risers 416a of the first coupon 402a. In at least some embodiments, the second bottom insulative substrate 602b defines cutouts 604b positioned to coincide with the positioning of the risers 416b of the second coupon 402b.

The insulative substrates 502a, 502b, 602a, and 602b are formed from any electrically-insulative materials suitable for implantation. In at least some embodiments, one or more of the insulative substrates 502a, 502b, 602a, and 602b is formed from a film. It may be advantageous to use a film to facilitate removal of air pockets between the insulative substrates 502a, 502b, 602a, and 602b. Films may also provide flexibility that facilitates the manufacturing process.

In at least some embodiments, one or more of the insulative substrates 502a, 502b, 602a, and 602b is formed from a material suitable for reflowing including, for example, one or more thermoplastics (e.g., Pellethane®, or the like). As will be discussed in more detail below, the micro-circuits and insulative substrates are formed into micro-circuit assemblies that are coupled to one another. Although the micro-circuits may be coupled to together using any suitable techniques (e.g., adhesives, or the like), it may be advantageous to re-flow the material of the insulative substrates to enable the multiple micro-circuit assemblies to form a more uniform, cohesive structure.

Turning to FIGS. 7A-7B, once the first and second coupons are sandwiched between the top and bottom insulative substrates, the first and second coupons and their respective top and bottom insulative substrates can be laminated to form micro-circuit assemblies. FIG. 7A is a schematic top perspective view of one embodiment of a first micro-circuit assembly 702a. The first micro-circuit assembly 702a includes the first coupon 402a laminated between the first top insulative substrate 502a and the first bottom insulative substrate 602a. FIG. 7B is a schematic top perspective view of one embodiment of a second micro-circuit assembly 702b. The second micro-circuit assembly 702b includes the second coupon 402b laminated between the second top insulative substrate 502b and the second bottom insulative substrate 602b.

In FIGS. 3A-15, the paddle body 304 is shown as being formed using two micro-circuit assemblies 702a and 702b. The paddle body 304, however, may include any suitable number of micro-circuit assemblies 702a and 702b including, for example one, two, three, four, five, six, seven, eight, or more micro-circuit assemblies 702a and 702b.

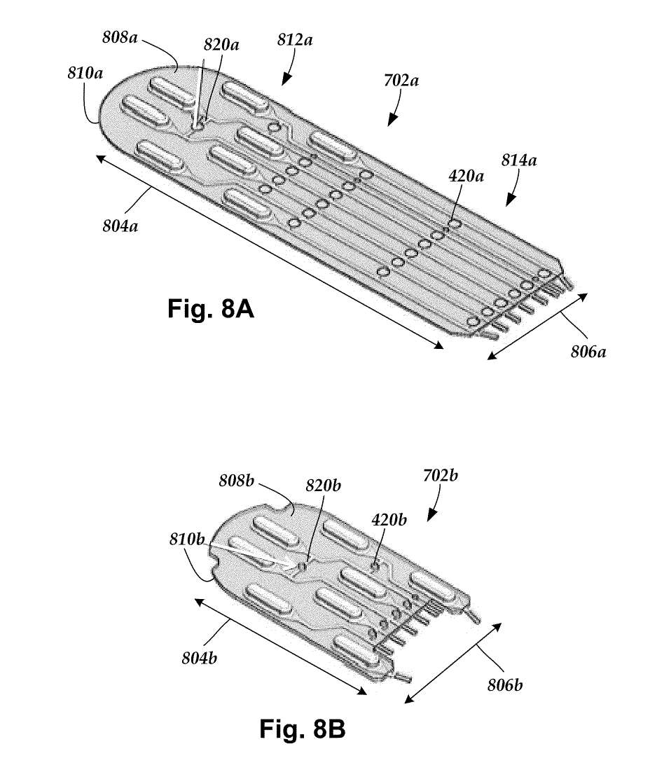

Turning to FIGS. 8A-8B, in at least some embodiments once the micro-circuits are laminated, the frames 424a and 424b can be removed and the conductor tie bars 420a and 420b coupling together adjacent micro-circuits can be severed. FIG. 8A is a schematic top perspective view of one embodiment of the first micro-circuit assembly 702a. FIG. 8B is a schematic top perspective view of one embodiment of the second micro-circuit assembly 702b.

As shown in FIG. 8A, the first micro-circuit assembly 702a has a length 804a, a width 806a, a first major surface 808a, an opposing second major surface 810a, a distal end portion 812a, and a proximal end portion 814a. In at least some embodiments, the frame 424a (see e.g., FIG. 7A) is removed from the first micro-circuit assembly 702a. In at least some embodiments, the conductor tie bars 420a (see e.g., FIG. 7A) are severed, thereby electrically-isolating each of the micro-circuits 336a (see e.g., FIG. 7A) from one another.

As shown in FIG. 8B, the second micro-circuit assembly 702b has a length 804b, a width 806b, a first major surface 808b and an opposing second major surface 810b. The frame 424b (see e.g., FIG. 7B) is removed from the second micro-circuit assembly 702b. The conductor tie bars 420b (see e.g., FIG. 7B) are severed, thereby electrically-isolating each of the micro-circuits 336b (see e.g., FIG. 7A) from one another.

The conductor tie bars 420a and 420b can be severed using any suitable technique including, for example, punching, laser cutting, drilling, or the like. In FIGS. 8A-8B (and in other figures) severed tie bars 420a and 420b are shown as circles. In at least some embodiments, when the conductor tie bars 420a and 420b are severed, one or more severed ends 820a and 820b of the conductor tie bars 420a and 420b, respectively, remain attached to at least some of the individual micro-circuits.

In at least some embodiments, the widths 806a and 806b of the first and second micro-circuit assemblies 702a and 702b are equal. In at least some embodiments, the length 804a of the first micro-circuit assembly 702a is greater than the length 804b of the second micro-circuit assembly 702b.

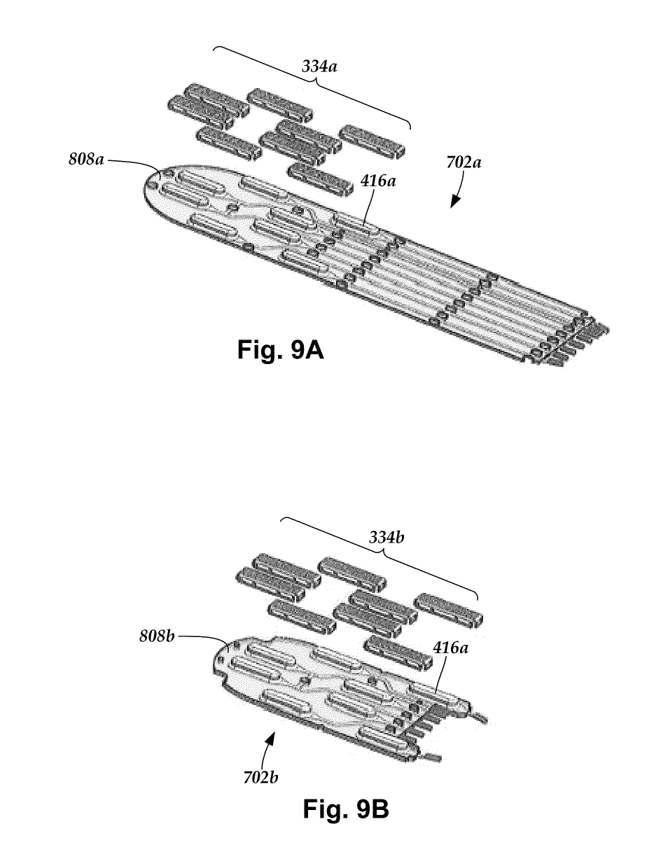

Turning to FIGS. 9A-9B, the electrodes 334a and 334b are coupled to the micro-circuit assemblies 702a and 702b, respectively. FIG. 9A is a schematic top perspective view of one embodiment of the first micro-circuit assembly 702a and the distal electrodes 334a. In FIG. 9A, the distal electrodes 334a are shown configured and arranged for disposing over the first major surface 808a of the first micro-circuit assembly 702a. FIG. 9B is a schematic top perspective view of one embodiment of the second micro-circuit assembly 702b and the proximal electrodes 334b. In FIG. 9B, the proximal electrodes 334b are shown configured and arranged for disposing over the first major surface 808b of the second micro-circuit assembly 702b.

In at least some embodiments, the electrodes 334a and 334b are configured and arranged for coupling directly to the risers 416a and 416b, respectively. In at least some embodiments, the distal electrodes 334a and 334b are fixtured and installed to the risers 416a and 416b, respectively. Accordingly, in at least some embodiments the configuration of the electrodes 334a and 334b along the paddle body 304 is determined by the configuration of the risers 416a and 416b along the micro-circuit assemblies 702a and 702b.

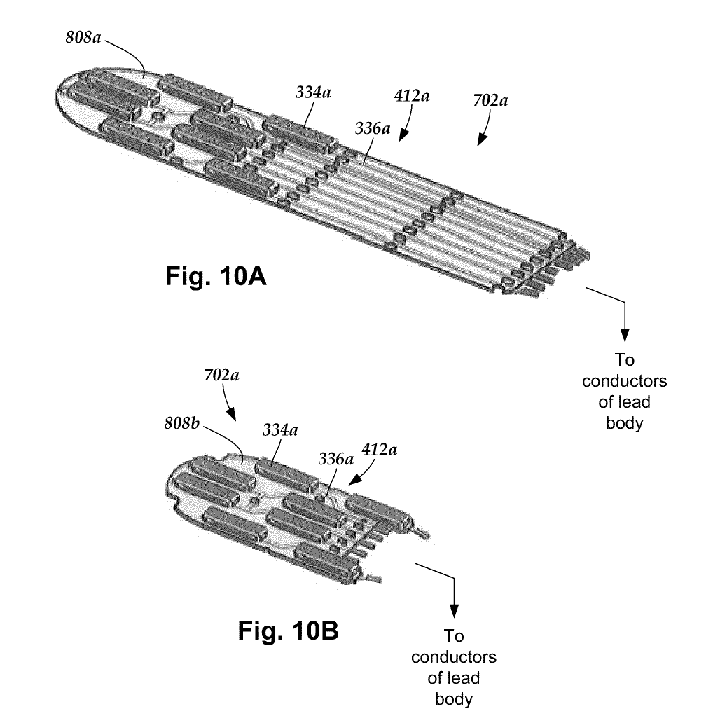

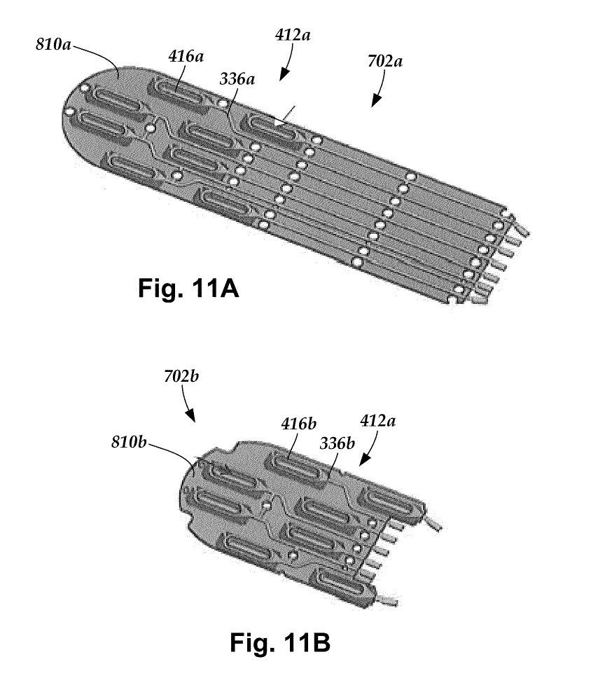

Turning to FIGS. 10A-11B, the electrodes are electrically coupled to their respective micro-circuits. FIG. 10A is a schematic top perspective view of one embodiment of the distal electrodes 334a mechanically coupled to the first major surface 808a of the first micro-circuit assembly 702a. FIG. 10B is a schematic top perspective view of one embodiment of the proximal electrodes 334b mechanically coupled to the first major surface 808b of the second micro-circuit assembly 702b.

FIG. 11A is a schematic bottom perspective view of one embodiment of the second major surface 810a of the first micro-circuit assembly 702a with the distal electrodes 334a mechanically coupled to the first major surface 808a. FIG. 11B is a schematic bottom perspective view of one embodiment of the second major surface 810b of the second micro-circuit assembly 702b with the proximal electrodes 334b mechanically coupled to the first major surface 808b.

As shown in FIGS. 11A-11B, the electrodes 334a and 334b are electrically coupled to the first ends 412a and 412b of the micro-circuits 336a and 336b, respectively. In at least some embodiments, the electrodes 334a and 334b are electrically coupled to the micro-circuits 336a and 336b, respectively, along the second major surfaces 810a and 810b of the micro-circuit assembly 702a and 702b, respectively. The electrodes 334a and 334b can be coupled to the micro-circuits 336a and 336b, respectively, in any suitable manner. In preferred embodiments, the electrodes 334a and 334b are laser welded to the micro-circuits 336a and 336b, respectively. It may be advantageous to laser weld the electrodes 334a and 334b to the micro-circuits 336a and 336b, respectively. Such a technique may provide a higher degree of reliability of the electrical connection than would otherwise be obtained using conventional techniques, such as blind, resistance welding.

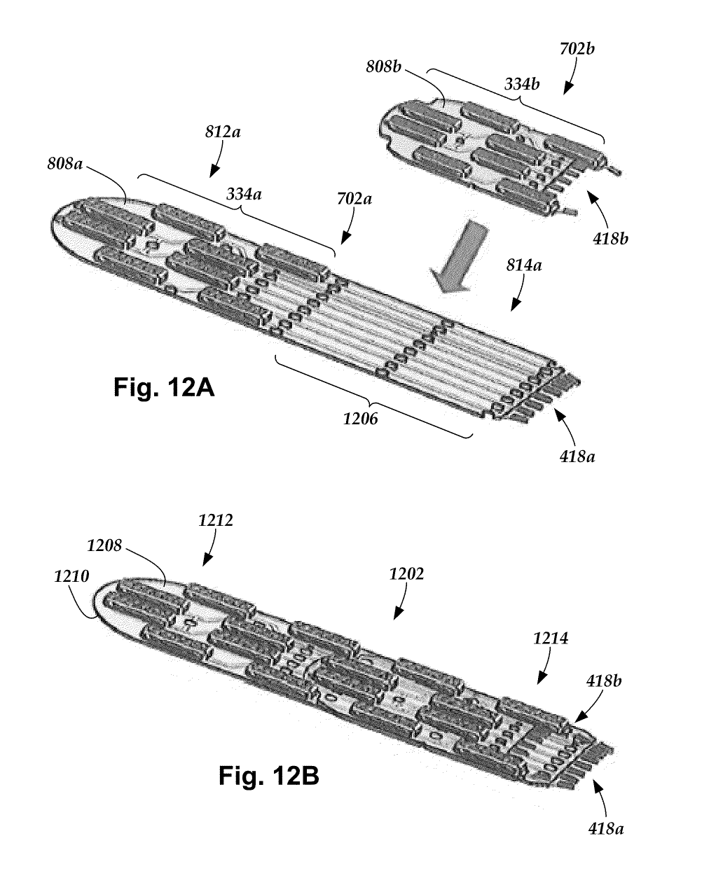

Turning to FIGS. 12A-12B, in at least some embodiments once the electrodes are mechanically coupled to the micro-circuit assemblies and electrically coupled to the micro-circuits of the micro-circuit assemblies, the micro-circuit assemblies are mechanically coupled to one another to form a composite micro-circuit assembly. FIG. 12A is a schematic perspective view of one embodiment of the first micro-circuit assembly 702a and the second micro-circuit assembly 702b, where the second micro-circuit assembly 702b is configured and arranged for disposing over a portion 1206 of the first major surface 808a of the first micro-circuit assembly 702a.

As mentioned above, in at least some embodiments the widths 806a and 806b of the first and second micro-circuit assemblies 702a and 702b are equal. In at least some embodiments, the length 804a of the first micro-circuit assembly 702a is greater than the length 804b of the second micro-circuit assembly 702b.

In at least some embodiments, the portion 1206 of the first major surface 808a of the first micro-circuit assembly 702a over which the second micro-circuit assembly 702b is disposed is proximal from the electrodes 334a along the first micro-circuit assemblies 702a. In at least some embodiments, the portion 1206 of the first major surface 808a of the first micro-circuit assembly 702a over which the second micro-circuit assembly 702b is disposed is positioned along the proximal end portion 814a of the first micro-circuit assemblies 702a. In at least some embodiments, the portion 1206 of the first major surface 808a of the first micro-circuit assembly 702a over which the second micro-circuit assembly 702b is disposed is recessed such that, when the second micro-circuit assembly 702b is disposed over the portion 1206, the first major surface 808a of the first micro-circuit assembly 702a is flush with the first major surface 808b of the second micro-circuit assembly 702b.

FIG. 12B is a schematic perspective view of one embodiment of the second micro-circuit assembly 702b disposed over the portion 1206 of the first major surface 808a of the first micro-circuit assembly 702a to form a composite micro-circuit assembly 1202. The composite micro-circuit assembly 1202 has a first major surface 1208, a second major surface 1210, a distal end portion 1212, and a proximal end 1412.

The composite micro-circuit assembly 1202 shown in FIG. 12B includes the electrical connectors 418a and 418b coupled to the micro-circuits 336a and 336b. When the second micro-circuit assembly 702b disposed over the first major surface 808a of the first micro-circuit assembly 702a, the micro-circuits 336a and 336b are configured into layers, with the micro-circuits 336a of the first micro-circuit assembly 702a being in a first layer, and with the micro-circuits 336b of the second micro-circuit assembly 702b being in a second layer over top of the first layer. In which case, the connectors 418a and 418b may be similarly arranged into layers with the connectors 418b disposed over the connectors 418b.

In at least some embodiments, the first micro-circuit assembly 702a and the second micro-circuit assembly 702b are coupled together. The first micro-circuit assembly 702a and the second micro-circuit assembly 702b may be coupled together in any suitable manner, such as by using one or more adhesives. In at least some embodiments, the first micro-circuit assembly 702a and the second micro-circuit assembly 702b are reflowed.

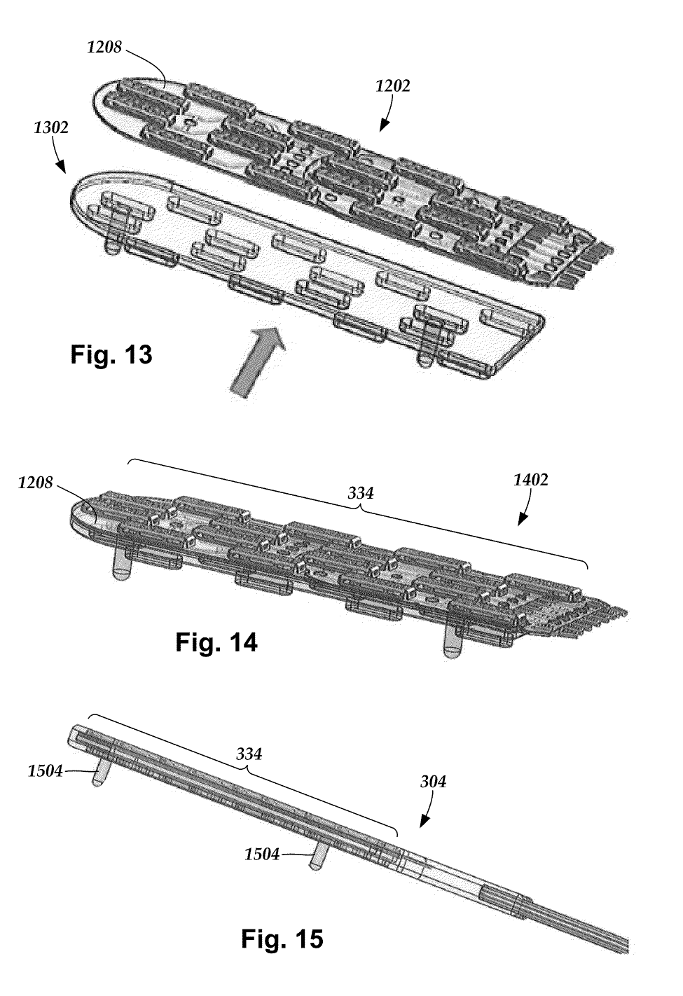

Turning to FIGS. 13-15, in at least some embodiments the composite micro-circuit assembly can be disposed over a molded carrier, re-flowed, and over-molded to form the paddle body 304. Additionally, the one or more lead bodies can be mechanically coupled to the paddle body and the conductive wires of the one or more lead bodies can be attached to the micro-circuits.

FIG. 13 is a schematic perspective view of one embodiment of the composite micro-circuit assembly 1202 and a carrier 1302. The composite micro-circuit assembly 1202 is configured and arranged for disposing over the carrier 1302. In at least some embodiments, of the composite micro-circuit assembly 1202 is configured and arranged for disposing over the carrier 1302 with the second major surface 1210 of the composite micro-circuit assembly 1202 abutting the carrier 1302. In at least some embodiments, the composite micro-circuit assembly 1202 is fixtured and installed to the carrier 1302.

The carrier 1302 can be formed from any electrically-insulative materials suitable for implantation. In at least some embodiments, the carrier 1302 is molded. In at least some embodiments, the carrier 1302 is formed from a material suitable for reflowing including, for example, one or more thermoplastics (e.g., Pellethane®, or the like).

FIG. 14 is a schematic perspective view of one embodiment of the composite micro-circuit assembly 1202 coupled to the carrier 1302 to form a pre-paddle body 1402 having the first major surface 1208 on which the electrodes 334 are disposed. The composite micro-circuit assembly 1202 can be coupled to the carrier 1302 in any suitable manner. In at least some embodiments, the composite micro-circuit assembly 1202 and the carrier 1302 are re-flowed to couple the composite micro-circuit assembly 1202 to the carrier 1302.

In at least some embodiments, the carrier 1302 is formed from the same material as one or more of the insulative substrates 502a, 502b, 602a, and 602b. It may be advantageous for the carrier 1302 and the insulative substrates 502a, 502b, 602a, and 602b to be formed from the same material to facilitate the coupling and uniformity of the carrier 1302 and the insulative substrates 502a, 502b, 602a, and 602b created during re-flowing process.

Once the composite micro-circuit assembly 1202 and the carrier 1302 are coupled together to form the pre-paddle body 1402, the pre-paddle body 1402 can be over-molded to form the paddle body 304 (see e.g., FIG. 3A). FIG. 15 is a schematic side view of one embodiment of the paddle body 304. In at least some embodiments, the paddle body 304 is formed by over-molding the pre-paddle body 1402. In at least some embodiments, the carrier 1302 includes one or more tooling bosses 1504. The tooling bosses 1504 may facilitate the over-molding process by providing a structure for positioning the pre-paddle body 1402 such that top surfaces of the electrodes 334 are disposed at a top surface of a mold used during the molding process. In embodiments of the pre-paddle body 1402 that utilize one or more tooling bosses 1504, the one or more tooling bosses 1504 can, preferably, be removed (e.g., detached from the paddle body 304, ground down flush with an outer surface of the paddle body 304, or the like) after the over-molding process is complete.

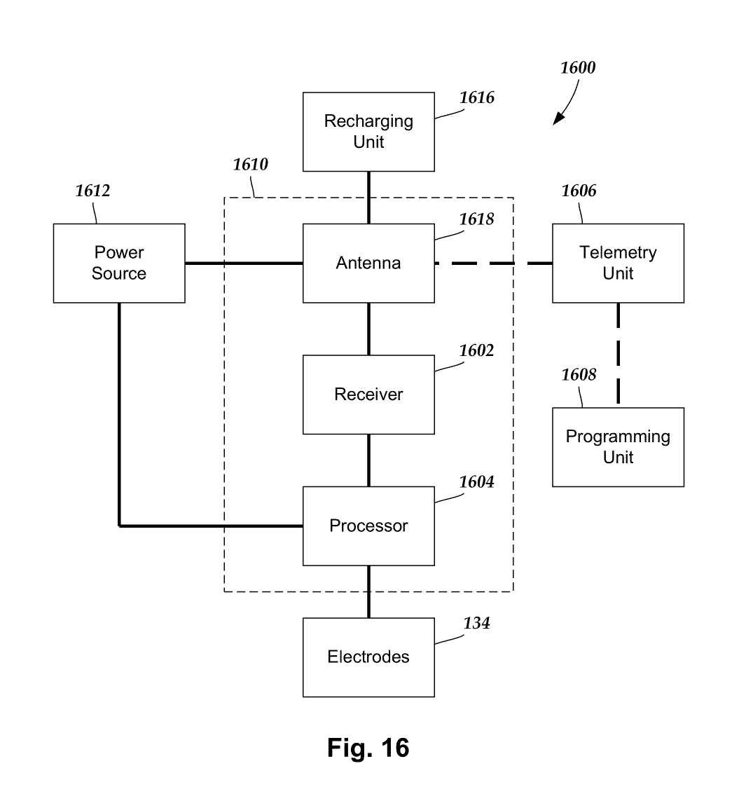

FIG. 16 is a schematic overview of one embodiment of components of an electrical stimulation system 1600 including an electronic subassembly 1610 disposed within a control module. It will be understood that the electrical stimulation system can include more, fewer, or different components and can have a variety of different configurations including those configurations disclosed in the stimulator references cited herein.

Some of the components (for example, power source 1612, antenna 1618, receiver 1602, and processor 1604) of the electrical stimulation system can be positioned on one or more circuit boards or similar carriers within a sealed housing of an implantable pulse generator, if desired. Any power source 1612 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1618 or a secondary antenna. The external power source can be in a device that is mounted on the skin of the user or in a unit that is provided near the user on a permanent or periodic basis.

If the power source 1612 is a rechargeable battery, the battery may be recharged using the optional antenna 1618, if desired. Power can be provided to the battery for recharging by inductively coupling the battery through the antenna to a recharging unit 1616 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 1604 is generally included to control the timing and electrical characteristics of the electrical stimulation system. For example, the processor 1604 can, if desired, control one or more of the timing, frequency, strength, duration, and waveform of the pulses. In addition, the processor 1604 can select which electrodes can be used to provide stimulation, if desired. In some embodiments, the processor 1604 may select which electrode(s) are cathodes and which electrode(s) are anodes. In some embodiments, the processor 1604 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1608 that, for example, allows modification of pulse characteristics. In the illustrated embodiment, the processor 1604 is coupled to a receiver 1602 which, in turn, is coupled to the optional antenna 1618. This allows the processor 1604 to receive instructions from an external source to, for example, direct the pulse characteristics and the selection of electrodes, if desired.

In one embodiment, the antenna 1618 is capable of receiving signals (e.g., RF signals) from an external telemetry unit 1606 which is programmed by a programming unit 1608. The programming unit 1608 can be external to, or part of, the telemetry unit 1606. The telemetry unit 1606 can be a device that is worn on the skin of the user or can be carried by the user and can have a form similar to a pager, cellular phone, or remote control, if desired. As another alternative, the telemetry unit 1606 may not be worn or carried by the user but may only be available at a home station or at a clinician's office. The programming unit 1608 can be any unit that can provide information to the telemetry unit 1606 for transmission to the electrical stimulation system 1600. The programming unit 1608 can be part of the telemetry unit 1606 or can provide signals or information to the telemetry unit 1606 via a wireless or wired connection. One example of a suitable programming unit is a computer operated by the user or clinician to send signals to the telemetry unit 1606.

The signals sent to the processor 1604 via the antenna 1618 and receiver 1602 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1600 to cease operation, to start operation, to start charging the battery, or to stop charging the battery. In other embodiments, the stimulation system does not include an antenna 1618 or receiver 1602 and the processor 1604 operates as programmed.

Optionally, the electrical stimulation system 1600 may include a transmitter (not shown) coupled to the processor 1604 and the antenna 1618 for transmitting signals back to the telemetry unit 1606 or another unit capable of receiving the signals. For example, the electrical stimulation system 1600 may transmit signals indicating whether the electrical stimulation system 1600 is operating properly or not or indicating when the battery needs to be charged or the level of charge remaining in the battery. The processor 1604 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation lead comprising:
a paddle body having a proximal end portion, a distal end portion, a first major surface, and an opposing second major surface, the paddle body comprising a plurality of micro-circuit assemblies, each of the plurality of micro-circuit assemblies comprising
a first electrically-nonconductive substrate,
a second electrically-nonconductive substrate, and
a plurality of micro-circuits laminated between the first electrically-nonconductive substrate and the second electrically-nonconductive substrate, the plurality of micro-circuits each having a first end portion and an opposing second end portion, wherein the second end portions of the plurality of micro-circuits are disposed at the proximal end portion of the paddle body;
a plurality of electrodes disposed on the paddle body, wherein the plurality of electrodes are electrically coupled to the first end portions of the plurality of micro-circuits;
at least one lead body having a distal end portion, a proximal end portion, and a longitudinal length, the distal end portions of each of the at least one lead body coupled to the proximal end portion of the paddle body;
a plurality of terminals disposed along the proximal end portion of each of the at least one lead body; and
a plurality of lead-body conductors coupled to the plurality of terminals and extending along the at least one lead body to the distal end portion of the at least one lead body, wherein the plurality of lead-body conductors are attached to the second end portions of the plurality of micro-circuits and electrically couple the plurality of terminals to the plurality of electrodes.

2. The electrical stimulation lead of claim 1, wherein, for each of the plurality of micro-circuit assemblies, at least one of the first electrically-nonconductive substrate or the second electrically-nonconductive substrate comprises a thermoplastic material.

3. The electrical stimulation lead of claim 1, wherein the plurality of micro-circuit assemblies each comprises a plurality of risers disposed at the first end portions of the plurality of micro-circuits.

4. The electrical stimulation lead of claim 3, wherein each of the plurality of electrodes is disposed over a different riser of the plurality of risers.

5. The electrical stimulation lead of claim 1, further comprising a carrier coupled to at least one of the plurality of micro-circuit assemblies.

6. The electrical stimulation lead of claim 5, wherein the carrier comprises at least one thermoplastic material.

7. The electrical stimulation lead of claim 1, wherein each of the plurality of micro-circuits comprises a different electrical connector disposed along the second end portion of that micro-circuit.

8. The electrical stimulation lead of claim 7, wherein the electrical connectors are arranged into at least two layers.

9. The electrical stimulation lead of claim 1, wherein a severed end of a conductor tie bar is attached to at least one of the plurality of micro-circuits.

10. The electrical stimulation lead of claim 1, wherein the plurality of micro-circuits comprise MP35N.

11. An electrical stimulating system comprising:
the electrical stimulation lead of claim 1;
a control module coupleable to the electrical stimulation lead, the control module comprising
a housing, and
an electronic subassembly disposed in the housing; and
a connector for receiving the electrical stimulation lead, the connector having a proximal end, a distal end, and a longitudinal length, the connector comprising
a connector housing defining a port at the distal end of the connector, the port configured and arranged for receiving the proximal end portion of at least one of the at least one lead body, and
a plurality of connector contacts disposed in the connector housing, the plurality of connector contacts configured and arranged to couple to at least one of the plurality of terminals disposed on the proximal end portion of the at least one lead body of the electrical stimulation lead.

12. A method of forming a paddle lead, the method comprising:
forming a plurality of micro-circuit assemblies, each of the plurality of micro-circuit assemblies comprising a plurality of micro-circuits laminated between electrically-nonconductive substrates, wherein each of the micro-circuits has a first end portion and an opposing second end portion;
mechanically coupling a plurality of electrodes to the plurality of micro-circuit assemblies;
electrically coupling the plurality of electrodes to the first end portions of the plurality of micro-circuits;
coupling together the plurality of micro-circuit assemblies and a carrier to form a paddle body;
extending a plurality of lead-body conductors along at least one lead body;
attaching the second end portions of the plurality of micro-circuits to the lead-body conductors; and
mechanically coupling the paddle body to the at least one lead body.

13. The method of claim 12, wherein mechanically coupling a plurality of electrodes to the plurality of micro-circuit assemblies comprises coupling the plurality of electrodes to a plurality of risers disposed in proximity to the first end portions of the plurality of micro-circuits.

14. The method of claim 12, wherein electrically coupling the plurality of electrodes to the first end portions of the plurality of micro-circuits comprises laser welding the plurality of electrodes to the plurality of micro-circuits.

15. The method of claim 12, wherein coupling together the plurality of micro-circuit assemblies and a carrier to form a paddle body comprises re-flowing at least one of material of the carrier or material of the electrically-nonconductive substrates.

16. The method of claim 12, wherein coupling together the plurality of micro-circuit assemblies and a carrier to form a paddle body comprises over-molding the paddle body.

17. The method of claim 16, wherein over-molding the paddle body comprises:
using tooling bosses coupled to the carrier to facilitate over-molding; and
removing the tooling bosses from the carrier after the over-molding is completed.

18. The method of claim 12, wherein attaching the second end portions of the plurality of micro-circuits to the lead-body conductors comprises crimping the micro-circuits to the lead-body conductors.

19. The method of claim 12, further comprising severing at least one conductor tie bar coupling together at least two of the plurality of micro-circuits.

20. An electrical stimulation lead, comprising:
a paddle body having a proximal end portion, a distal end portion, a first major surface, and an opposing second major surface, the paddle body comprising a plurality of micro-circuit assemblies, the plurality of micro-circuit assemblies comprising a first micro-circuit assembly and a second micro-circuit assembly stacked over top of a portion of the first micro-circuit assembly, each of the plurality of micro-circuit assemblies comprising
a first electrically-nonconductive substrate,
a second electrically-nonconductive substrate, and
a plurality of micro-circuits laminated between the first electrically-nonconductive substrate and the second electrically-nonconductive substrate;
a plurality of electrodes disposed on the paddle body, wherein the plurality of electrodes are electrically coupled to the plurality of micro-circuits;
at least one lead body having a distal end portion, a proximal end portion, and a longitudinal length, the distal end portions of each of the at least one lead body coupled to the paddle body;
a plurality of terminals disposed along the proximal end portion of each of the at least one lead body; and
a plurality of lead-body conductors coupled to the plurality of terminals and extending along the at least one lead body to the distal end portion of the at least one lead body, wherein the plurality of lead-body conductors are attached to the plurality of micro-circuits and electrically couple the plurality of terminals to the plurality of electrodes.
